(19) United States
(12) Patent Application Publication
(10) Pub. No.: US 2014/0148822 A1
(43) Pub. Date: May 29, 2014

(54) METHOD AND APPARATUS FOR POLISHING HUMAN SKIN

(71) Applicant: Nahayan Ameen Abdulla Ibrahim Al Mahza, Khalifa City A (AE)

(72) Inventor: Nahayan Ameen Abdulla Ibrahim Al Mahza, Khalifa City A (AE)

(21) Appl. No.: 13/684,597

(22) Filed: Nov. 26, 2012

(51) Int. Cl. A61B 17/3205

(52) U.S. Cl. CPC A61B 17/3205
USPC 606/131

(57) ABSTRACT

The embodiments herein provide a skin polishing apparatus and a method for cleaning and removing a dead skin and dirt from a human body and for polishing a human skin. The apparatus has a skin abrading head provided with a removable brush, which is rotated to remove the dust and to polish the skin. The skin abrading head is mounted with two shower gel tanks filled with a cleansing liquid and a perfume which are sprayed on to the skin to clean and remove the dead skin and the dirt and to polish the skin with a fragrance during a rotation of the head and brush. The apparatus is powered by the rechargeable batteries. The abrading head is rotated with a motor. The rotating speed of the abrading head is controlled with a printed circuit board to a high or low rotating speed.

### BACKGROUND

1. Technical Field

The embodiments herein generally relate to a polishing apparatus and particularly relate to a skin polishing apparatus. The embodiments herein more particularly relate to a method and apparatus for cleaning and removing a dead skin and dirt from a human body and polishing a human skin.

2. Description of the Related Art

The structure of the human skin is divided into two layers, the epidermis and the dermis. The layer at the very surface of the skin is called the Stratum corneum. This layer mainly consists of the dead skin cells. A dead skin has a way of making the human skin look dull and lacking in luster. It is necessary to remove the dead skin from the human body to reveal the new grown healthy cells that are beneath. The dead skin cells act as a barrier limiting in absorbing the nutrients from the nourishing creams. Also, a removal of the dead skin cells is necessary to keep ourselves away from a number of different issues such as the formation of thick skins etc. A removal of the dead skin cells from the face may help to reveal a smoother and a more youthful skin.

One of the prior art methods provide an electrically powered scrubbing device adapted to gently and painlessly remove the keratinized epidermal portions of the hands and the feet. A manual speed selector is provided to allow the user to manually select a variety of RPM motor speeds for scrubbing the epidermal portions. The manual speed selector has multiple settings such as a low, a medium, and a high that are selected by a user to designate a preferred speed. The major drawback of this prior art is that it is limited for scrubbing only the epidermal portions of the hands and the feet.

The epidermal portions of the face cannot be scrubbed. The user has to manually apply a cleansing gel onto the skin after removing the keratinized epidermal portions of the hands and the feet.

Another prior art method provides a tissue removal device for removing the dead and old skin cells on an epidermis layer of the human skin to provide a revitalized and a fresh skin surface. The abrading head of the tissue removal device is replaceable individually such that the user is facilitated to select a proper abrasion head to effectively remove the cells on treating the skin surface. The major drawback of this prior art is that the user has to manually apply a shower gel for an easy removal of the dead skin cells.

Hence there exists a need for a method and apparatus that overcomes the above mentioned drawbacks by providing a skin polishing apparatus for cleaning and removing a dead skin and a dirt from a human body and polishing a human skin. Further, there is a need for a skin polishing apparatus with removable brush heads that are interchangeable for use in different parts like face, body, feet etc. Further, there is also a need for a skin polishing apparatus that comprises two shower gel tanks for filling a desired cleansing gel and a perfume and also for dispensing it onto a human skin while abrading a desired area of the human skin.

The abovementioned shortcomings, disadvantages and problems are addressed herein and which will be understood by reading and studying the following specification.

### OBJECTS OF THE EMBODIMENTS

The primary object of the embodiments herein is to provide a skin polishing apparatus for cleaning and removing a dead skin and dirt from a human body and polishing a human skin.

Another object of the embodiments herein is to provide a skin polishing apparatus comprising the removable brush heads, which are interchangeable, so that each removable brush head having a plurality of parameters such as size, speed and a bristle type is used.

Yet another object of the embodiments herein is to provide a skin polishing apparatus that comprises two shower gel tanks for filling a desired cleansing gel and a perfume and also dispensing it onto a human skin while abrading the skin.

These and other objects and advantages of the embodiments herein will become readily apparent from the following detailed description taken in conjunction with the accompanying drawings.

### SUMMARY

The various embodiments herein provide a skin polishing apparatus for cleaning and removing a dead skin and dirt from a human body and polishing a human skin. The skin polishing apparatus comprises a skin abrading head. A removable brush head is provided at a front portion of the skin abrading head. The removable brush comprises a plurality of bristles projected outwardly from an outer peripheral surface for abrading a human skin. A first shower gel tank and a second shower gel tank are arranged at a back portion of the skin abrading head for storing a desired cleansing liquid and a perfume respectively. A first press-button release mechanism is mounted on a top portion of the first shower gel tank for releasing the cleansing liquid and a second press-button release mechanism is mounted on a top portion of the second shower gel tank for releasing the perfume onto the human skin. A liquid outlet is arranged above the removable brush head at a front portion of the skin abrading head for dispensing the cleansing liquid from the first shower gel tank and the perfume from the second shower gel tank respectively. The skin polishing apparatus comprises a pair of inbuilt rechargeable batteries for storing and supplying an electrical power to the skin abrading head. Further, a user fills the cleansing liquid and the perfume in the first shower gel tank and the second shower gel tank respectively and pushes the first press-button and the second press-button to release the cleansing gel and the perfume from the first shower gel tank and the second shower gel tank respectively from a central portion of the removable brush to clean and remove a dead skin, a dirt from a human body and polish a skin during a rotation of the skin abrading head.

According to an embodiment herein, the skin polishing apparatus further comprises a handle that is integrally formed with a built-in grip for providing a grip to a user.

According to an embodiment herein, a power switch is provided on a front portion of the handle for actuating the skin abrading head. A power indicator light is provided on the front portion of the handle below the power switch for indicating a status of the skin abrading head and wherein the status includes an ON/OFF condition of the skin abrading head.

According to an embodiment herein, a charging connector is provided on a back portion of the handle for connecting the skin polishing apparatus to a main supply.

According to an embodiment herein, a printed circuit board (PCB) is mounted along a central axis of the handle for controlling the ON/OFF condition of the device and a rotatory motion of the removable brush head of the skin polishing apparatus for regulating a rotating speed of the removable brush head into a high speed or to a low speed.

According to an embodiment herein, a motor is mounted along a central axis of the handle below the PCB. The motor is driven by a power from the rechargeable batteries. A driving gear is mounted along a central axis of the motor for conveying a rotary motion of the motor to a first driven gear and a second driven gear.

According to an embodiment herein, the first driven gear and the second driven gear is connected to a driving gear to regulate the rotary speed of a rotator shaft. The rotator shaft is extended from the first driven gear to the skin abrading head for actuating the removable brush head.

According to an embodiment herein, the removable brush head is interchangeable and each removable brush head includes a plurality of parameters. The plurality of parameters includes a size, a speed and a bristle type.

According to an embodiment herein, a shape of the removable brush head is any one of a circular shape, an oval shape, an elliptical shape, and a spherical shape.

According to an embodiment herein, the cleansing liquid comprises a skin polishing gel selected from a group consisting of a shower gel, a deep cleansing gel, a sunscreen lotion, a cream, and a combination thereof

According to an embodiment herein, the first shower gel tank and the second shower gel tank are transparent.

According to an embodiment herein, the skin abrading head has waterproof characteristics.

The various embodiments herein provide a method for cleaning and removing a dead skin and dirt from a human body and polishing a human skin. The method comprises the steps of filling a first shower gel tank in a skin abrading head with a cleansing liquid, filling a second shower gel tank in the skin abrading head with a perfume, actuating the skin abrading head by switching ON a power switch, pressing a first press-button release mechanism for releasing the cleansing liquid onto a human skin during a rotation of a rotatable brush head provided at the skin abrading head, pressing a second press-button release mechanism for releasing the perfume onto the human skin and running the skin abrading head in a specific area of the skin for removing a dead skin, a dirt and polishing the skin.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a top perspective view of the skin polishing apparatus, according to an embodiment herein.

FIG. 2 illustrates an exploded perspective view of the skin polishing apparatus, according to an embodiment herein.

FIG. 3 illustrates a side perspective view of the skin polishing apparatus with a tank cover assembled condition, according to an embodiment herein.

FIG. 4 illustrates a left side view of the skin polishing apparatus with a tank cover removed condition, according to an embodiment herein.

FIG. 5A illustrates a front perspective view of the skin polishing apparatus without a front cover according to an embodiment herein.

FIG. 5B illustrates a perspective view of a front cover of the skin polishing apparatus, according to an embodiment herein.

FIG. 6 illustrates a front side sectional view of the skin polishing apparatus showing the internal components, according to an embodiment herein.

FIG. 7 illustrates a left side sectional view of the skin polishing apparatus, according to an embodiment herein.

FIG. 8 illustrates a vertical side view of the skin polishing apparatus, according to an embodiment herein.

FIG. 9 illustrates a bottom view of the skin polishing apparatus, according to an embodiment herein.

FIG. 10 illustrates a backside view of the skin polishing apparatus, according to an embodiment herein.

FIG. 11 illustrates a topside view of the skin polishing apparatus, according to an embodiment herein.

FIG. 12 illustrates a front view of a hanger mounted on a wall for holding the skin polishing apparatus, according to an embodiment herein.

FIG. 13 illustrates a vertical side perspective view of the skin polishing apparatus hanged to the wall with the help of an external accessory, according to an embodiment herein.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a skin polishing apparatus for cleaning and removing a dead skin and dirt from a human body and polishing a human skin. The skin polishing apparatus comprises a skin abrading head. A removable brush head is provided at a front portion of the skin abrading head. The removable brush comprises a plurality of bristles projected outwardly from an outer peripheral surface for abrading a human skin. A first shower gel tank and a second shower gel tank are arranged at a back portion of the skin abrading head for storing a desired cleansing liquid and a perfume respectively. A first press-button release mechanism is mounted on a top portion of the first shower gel tank for releasing the cleansing liquid and a second press-button release mechanism is mounted on a top portion of the second shower gel tank for releasing the perfume onto the human skin. A liquid outlet is arranged above the removable brush head at a front portion of the skin abrading head for dispensing the cleansing liquid from the first shower gel tank and the perfume from the second shower gel tank respectively. The skin polishing apparatus comprises a pair of inbuilt rechargeable batteries for storing and supplying an electrical power to the skin abrading head. Further, a user fills the cleansing liquid and the perfume in the first shower gel tank and the second shower gel tank respectively and pushes the first press-button and the second press-button to release the cleansing gel and the perfume from the first shower gel tank and the second shower gel tank respectively from a central portion of the removable brush to clean and remove a dead skin, a dirt from a human body and polish a skin during a rotation of the skin abrading head.

According to an embodiment herein, the skin polishing apparatus further comprises a handle that is integrally formed with a built-in grip for providing a grip to a user.

According to an embodiment herein, a power switch is provided on a front portion of the handle for actuating the skin abrading head. A power indicator light is provided on the front portion of the handle below the power switch for indicating a status of the skin abrading head and wherein the status includes an ON/OFF condition of the skin abrading head.

According to an embodiment herein, a charging connector is provided on a back portion of the handle for connecting the skin polishing apparatus to a main supply.

According to an embodiment herein, a printed circuit board (PCB) is mounted along a central axis of the handle for controlling the ON/OFF condition of the device and a rotatory motion of the removable brush head of the skin polishing apparatus for regulating a rotating speed of the removable brush head into a high speed or to a low speed.

According to an embodiment herein, a motor is mounted along a central axis of the handle below the PCB. The motor is driven by a power from the rechargeable batteries. A driving gear is mounted along a central axis of the motor for conveying a rotary motion of the motor to a first driven gear and a second driven gear.

According to an embodiment herein, the first driven gear and the second driven gear is connected to a driving gear to regulate the rotary speed of a rotator shaft. The rotator shaft is extended from the first driven gear to the skin abrading head for actuating the removable brush head.

According to an embodiment herein, the removable brush head is interchangeable and each removable brush head includes a plurality of parameters. The plurality of parameters includes a size, a speed and a bristle type.

According to an embodiment herein, a shape of the removable brush head is any one of a circular shape, an oval shape, an elliptical shape, and a spherical shape.

According to an embodiment herein, the cleansing liquid comprises a skin polishing gel selected from a group consisting of a shower gel, a deep cleansing gel, a sunscreen lotion, a cream, and a combination thereof

According to an embodiment herein, the first shower gel tank and the second shower gel tank are transparent.

According to an embodiment herein, the skin abrading head has waterproof characteristics.

The various embodiments herein provide a method for cleaning and removing a dead skin and dirt from a human body and polishing a human skin. The method comprises the steps of filling a first shower gel tank in a skin abrading head with a cleansing liquid, filling a second shower gel tank in the skin abrading head with a perfume, actuating the skin abrading head by switching ON a power switch, pressing a first press-button release mechanism for releasing the cleansing liquid onto a human skin during a rotation of a rotatable brush head provided at the skin abrading head, pressing a second press-button release mechanism for releasing the perfume onto the human skin and running the skin abrading head in a specific area of the skin for removing a dead skin, a dirt and polishing the skin.

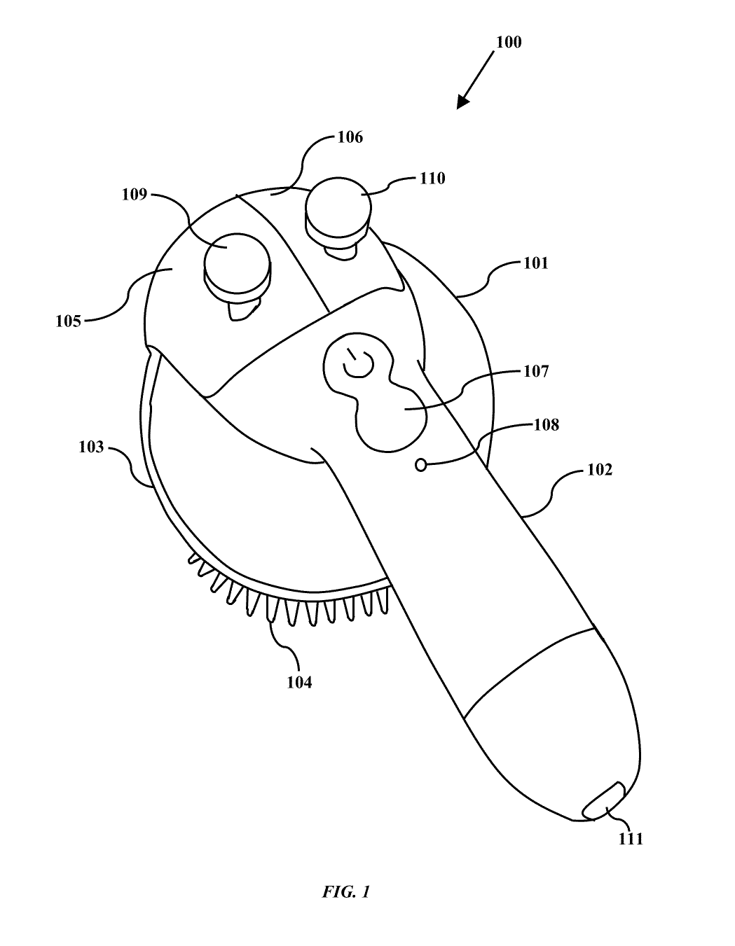

FIG. 1 illustrates a top perspective view of the skin polishing apparatus, according to an embodiment herein. The skin polishing apparatus 100 for cleaning and removing dead skin and dirt from the human body and polishing the human skin comprises a skin abrading head 101 and a handle 102. The skin abrading head 101 comprises a removable brush head 103 at a front portion, a first shower gel tank 105 and a second shower gel tank 106 at a back portion as shown in FIG. 1. A plurality of bristles 104 is projected outwardly from the outer peripheral surface of the removable brush head 103. The handle 102 comprises a power switch 107 and a power indicator light 108 at a front portion and a charging connector at a back portion 111. A first press-button mechanism 109 is mounted on a top portion of the first shower gel tank 105 for dispensing a cleansing liquid onto a human skin and similarly a second press-button mechanism 110 is mounted on the top portion of the second shower gel tank 106 for dispensing a perfume onto the human skin.

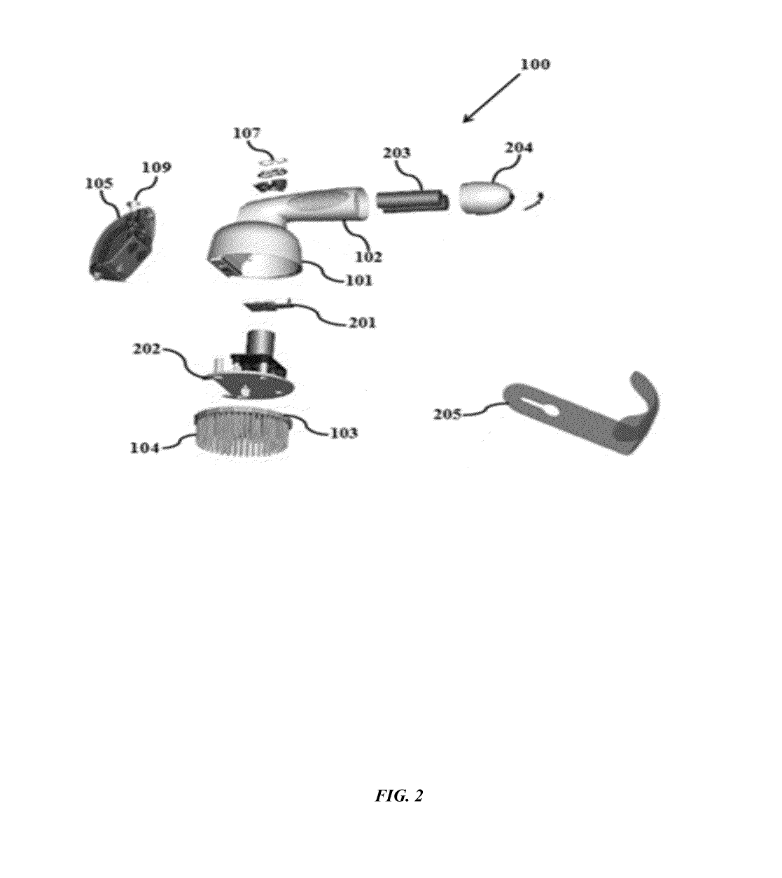

FIG. 2 illustrates an exploded perspective view of the skin polishing apparatus, according to an embodiment herein. The internal structure of the skin polishing apparatus 100 comprises a motor 202 electronically coupled to and controlled by a printed circuit board (PCB) 201. The motor 202 and the PCB 201 are electrically coupled to a pair of rechargeable batteries 203. The rechargeable batteries 203 are placed internally in the space inside the handle 102. The rechargeable batteries 203 are covered externally by a battery cover 204 provided with a silicon seal ring and a charging connector. A removable brush head 103 is mounted below the motor 202. A plurality of bristles 104 is projected outwardly from the outer peripheral surface of the removable brush head 103. A first shower gel tank 105 and a second shower gel tank are arranged at a front portion of the skin abrading head 101. A first press-button mechanism 109 is mounted on a top portion of the first shower gel tank 105 for dispensing a cleansing liquid onto a human skin and similarly a second press-button mechanism is mounted on the top portion of the second shower gel tank for dispensing a perfume onto the human skin. A power switch 107 is provided to switch ON/OFF the polishing head. A hanger 205 is provided to mount the polishing apparatus on a wall when not in use.

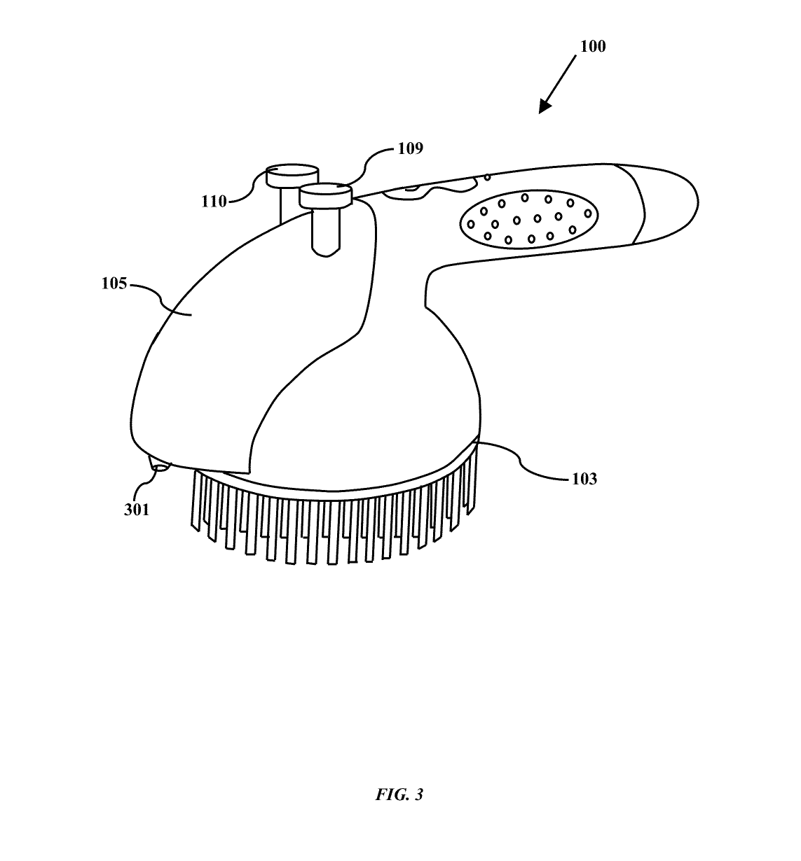

FIG. 3 illustrates a side perspective view of the skin polishing apparatus with a tank cover in closed condition, according to an embodiment herein. The first shower gel tank 105 stores a cleansing liquid. The second shower gel tank stores a perfume. A liquid outlet 301 is arranged above the removable brush head 103 at the front portion of the skin abrading head. The cleansing liquid and the perfume are dispensed through the liquid outlet 301 onto the human skin from the first shower gel tank 105 and the second shower gel tank when the user pushes/activates the first press-button mechanism 109 and the second press-button mechanism 110 respectively. The cleansing liquid and the perfume are dispensed from the liquid outlet 301 while the skin polishing apparatus 100 is rotated to clean and remove the dead skin and also to polish the human skin with an amazing fragrance in the skin.

According to an embodiment herein, the cleansing liquid is any one of a skin polishing gel selected from a group consisting of a shower gel, a deep cleansing gel, a sunscreen agent, cream, and a sun lighting agent etc.

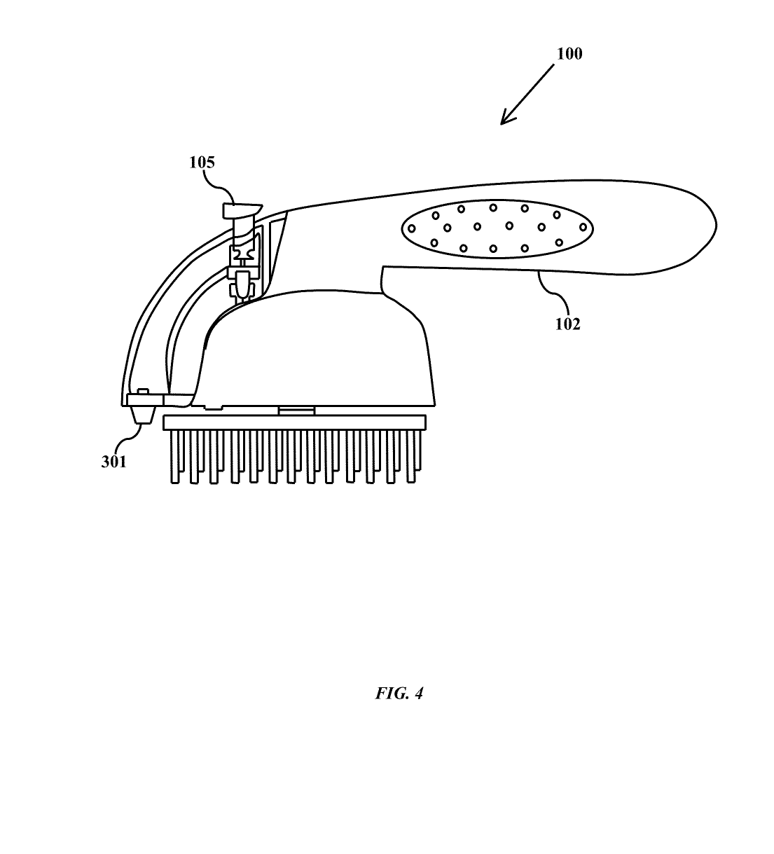

FIG. 4 illustrates a left side view of the skin polishing apparatus with a front cover removed condition, according to an embodiment herein. The first shower gel tank stores a cleansing liquid. The second shower gel tank stores a perfume. A liquid outlet 301 is arranged above the removable brush head at the front portion of the skin abrading head. The cleansing liquid and the perfume are dispensed through the liquid outlet 301 onto the human skin from the first shower gel tank and the second shower gel tank when the user pushes/activates the first press-button mechanism 109 and the second press-button mechanism respectively. The cleansing liquid and the perfume are dispensed from the liquid outlet 301 while the skin polishing apparatus 100 is rotated to clean and remove dead skin and also to polish the human skin with an amazing fragrance in the skin. The handle 102 is provided with a built-in grip for providing a comfortable grip to a user.

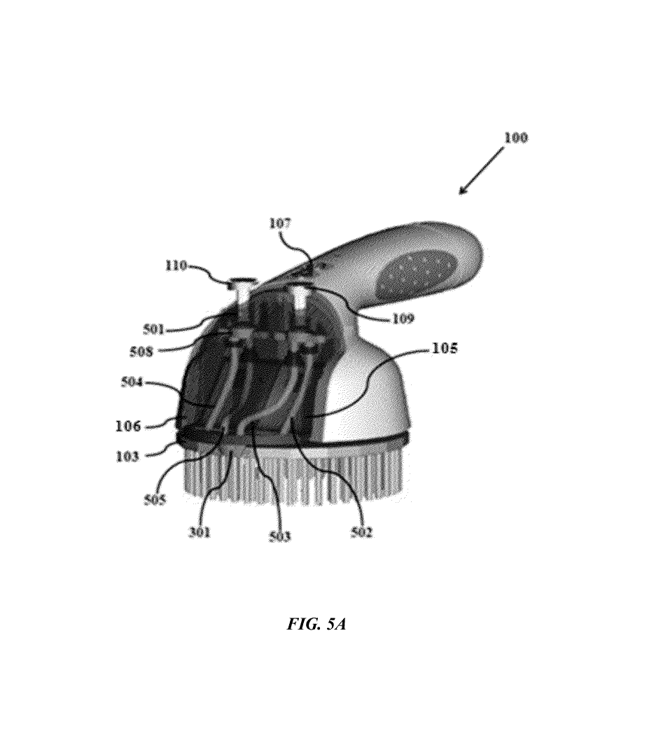

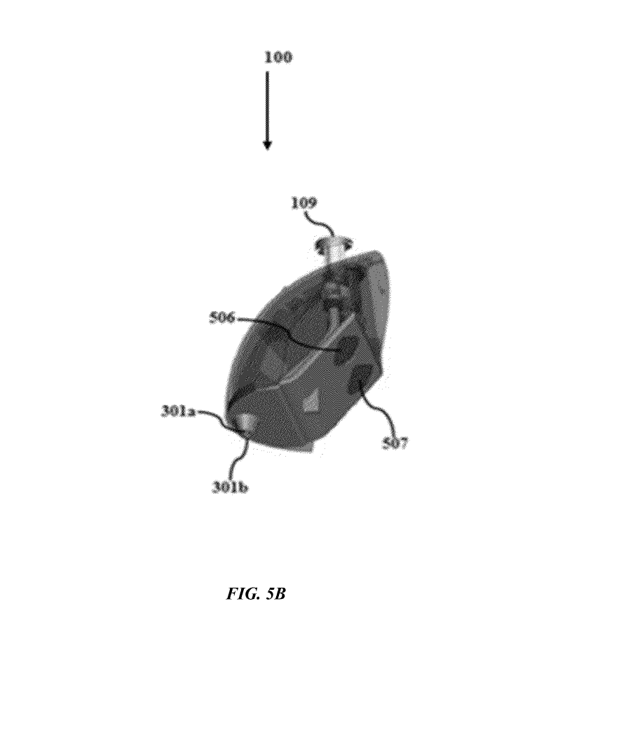

FIG. 5A illustrates a front side perspective view of the skin polishing apparatus without the front cover according to an embodiment herein and FIG. 5B illustrates a perspective view of the front cover of the skin polishing apparatus, according to an embodiment herein. The skin abrading head of the skin polishing apparatus 100 comprises a first shower gel tank 105 for storing the cleansing liquid and a second shower gel tank 106 for storing the perfume. The first shower gel tank 105 dispenses the cleansing liquid and the second shower gel tank 106 dispenses the perfume onto the human skin when needed. The first press-button mechanism 109 is mounted on the top portion of the first shower gel tank 105 and the second press-button mechanism 110 is mounted on the top portion of the second shower gel tank 106 respectively. The first shower gel tank 105 comprises a first liquid inlet tube 502 and a first liquid outlet tube 503. Similarly, the second shower gel tank 106 comprises a second liquid inlet tube 504 and a second liquid outlet tube 505. The liquid outlet 301 is attached above the removable brush head 103 at the front portion of the skin abrading head. The liquid outlet 301 further comprises a first outlet 301a for dispensing the cleansing liquid from the first shower gel tank 105 and a second outlet 301b for dispensing the perfume from the second shower gel tank 106. One end of the first liquid inlet tube 502 is inserted in to the first shower gel tank 105 for sucking the cleansing liquid and the other end is connected to a piston room 508. One end of the first liquid outlet tube 503 is connected to the piston room 508 and the other end is connected to the first outlet 301a for dispensing the cleansing liquid onto human skin through the liquid outlet 301. Similarly one end of the second liquid inlet tube 504 is inserted in to the second shower gel tank 106 for sucking the perfume and the other end is connected to a piston room 508. One end of the second liquid outlet tube 505 is connected to piston room 508 and the other end is connected to the second outlet 301b for dispensing the perfume onto human skin the liquid outlet 301.

When the user pushes/activates the first press-button mechanism 109 or the second press-button mechanism 110, a pressure is applied on the piston 501 which forces the respective liquid inlet tubes of the respective shower gel tanks to suck the respective liquids. The sucked liquid is sent to the respective liquid outlet tubes to dispense the respective liquid onto the human skin.

According to an embodiment herein, the desired cleansing liquid is filled to the first shower gel tank 105 and the second shower gel tank 106 through a first liquid inlet port 506 and a second liquid inlet port 507 respectively. A power switch 107 is provided on the handle to switch ON/OFF the polishing apparatus 100.

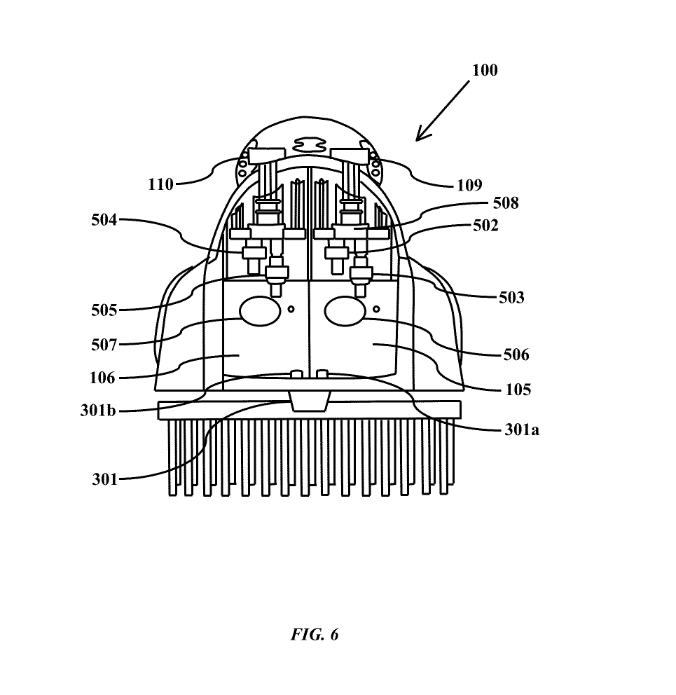

FIG. 6 illustrates a front side sectional view of the skin polishing apparatus, according to an embodiment herein. The skin abrading head of the skin polishing apparatus 100 comprises a first shower gel tank 105 for storing the cleansing liquid and a second shower gel tank 106 for storing the perfume. The first shower gel tank 105 dispenses the cleansing liquid and the second shower gel tank 106 dispenses the perfume onto the human skin whenever needed. The first press-button mechanism 109 is mounted on the top portion of the first shower gel tank 105 and the second press-button mechanism 110 is mounted on the top portion of the second shower gel tank 106 respectively. The first shower gel tank 105 comprises a first liquid inlet tube 502 and a first liquid outlet tube 503. Similarly, the second shower gel tank 106 comprises a second liquid inlet tube 504 and a second liquid outlet tube 505. The liquid outlet 301 is attached above the removable brush head at the front portion of the skin abrading head. The liquid outlet 301 further comprises a first outlet 301a for dispensing the cleansing liquid from the first shower gel tank 105 and a second outlet 301b for dispensing the perfume from the second shower gel tank 106. One end of the first liquid inlet tube 502 is inserted into the first shower gel tank 105 for sucking the cleansing liquid and the other end is connected to a piston room 508. One end of the first liquid outlet tube 503 is connected to the piston room 508 and the other end is connected to the first outlet 301a for dispensing the cleansing liquid onto human skin through the liquid outlet 301. Similarly, one end of the second liquid inlet tube 504 is inserted into the second shower gel tank 106 for sucking the perfume and the other end is connected to a piston room 508. One end of the second liquid outlet tube 505 is connected to piston room 508 and the other end is connected to the second outlet 301b for dispensing the perfume onto human skin the liquid outlet 301.

When the user pushes/activates the first press-button mechanism 109 or the second press-button mechanism 110, a pressure is applied on the piston which forces the respective liquid inlet tubes of the respective shower gel tanks to suck the respective liquids. The sucked liquid is sent to the respective liquid outlet tubes to dispense the respective liquid onto the human skin.

The desired cleansing liquid is filled to the first shower gel tank 105 and the second shower gel tank 106 through a first liquid inlet port 506 and a second liquid inlet port 507 respectively.

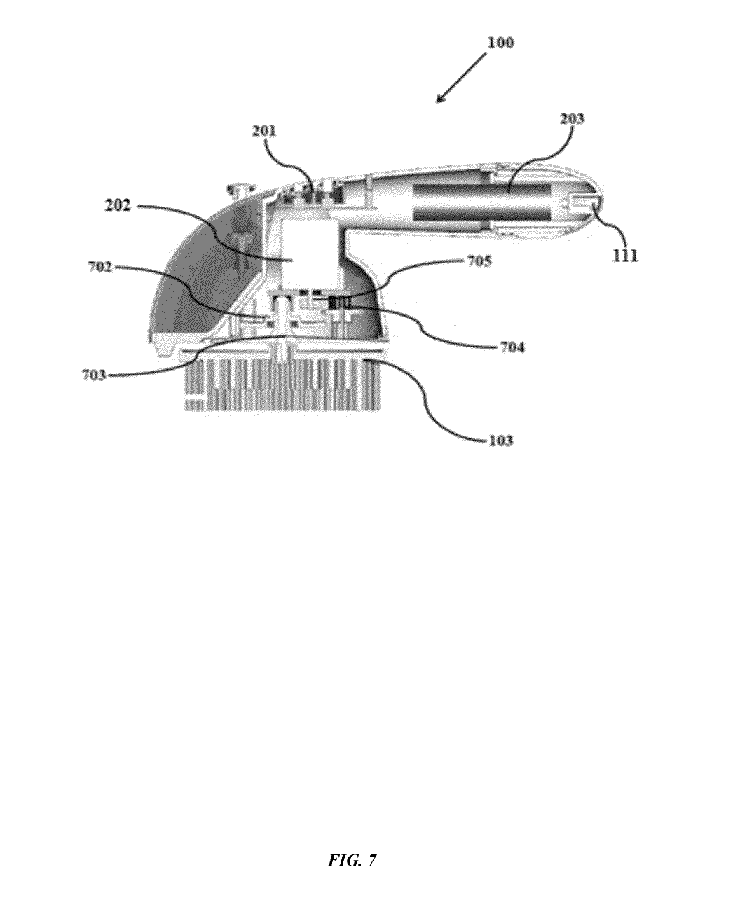

FIG. 7 illustrates a left side sectional view of the skin polishing apparatus, according to an embodiment herein. The skin polishing apparatus 100 comprises a printed circuit board (PCB) 201 adopted at the front portion of the handle and a motor 202 is electrically coupled to the PCB 201. The PCB 201 and the motor 202 are mounted one below the other. The PCB 201 and the motor 202 are electrically coupled to a pair of batteries 203. The batteries 203 are rechargeable. A driving gear 705 is arranged along a central axis of the motor 202. The driving gear 705 is further connected a first driven gear 702 and a second driven gear 704 to regulate the rotary speed of a rotator shaft 703. The rotator shaft 703 is further connected to the removable brush head 103, which is placed around the rotator shaft 703. A charging connector 111 is provided on the back portion of the handle for connecting the skin polishing apparatus 100 to the main power supply.

The charging connector 111 supplies the adequate electrical power to the rechargeable batteries 203 when connected to the main power supply. The rechargeable batteries 203 stores the electrical power received from the main power supply. When the motor 202 is actuated, the rechargeable batteries 203 drive the motor 202 from the stored electrical power, which in-turn drives the driving gear 705. Further, the driving gear 705 transmits the rotary motion to the first driven gear 702 and the second driven gear 704 respectively. The first driven gear 702 and the second driven gear 704 regulate the rotary speed of the rotator shaft 703. The removable brush head 103 arranged around the rotator shaft 703 also rotates to make the bristles to clean and remove dead skin and dirt from the human body and polish the human skin. The PCB 201 controls the ON/OFF and the rotatory motion of the removable brush head 103 of the skin polishing apparatus 100 to high/low speed.

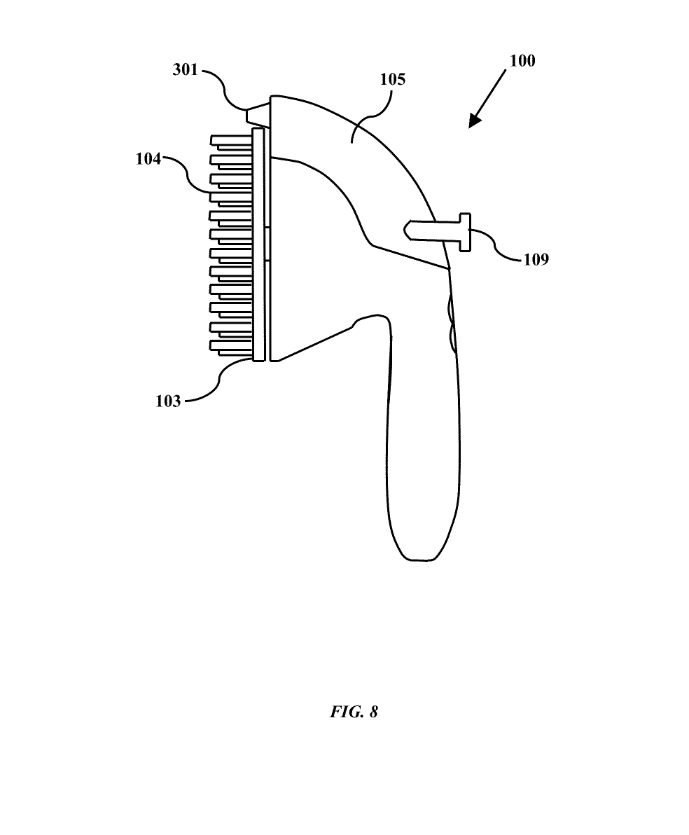

FIG. 8 illustrates a vertical side view of the skin polishing apparatus, according to an embodiment herein. A plurality of bristles 104 is projected outwardly from the outer peripheral surface of the removable brush head 103. The liquid outlet 301 is arranged above the removable brush head 103 at the front portion of the skin polishing apparatus 100. The cleansing liquid and the perfume are dispensed through the liquid outlet 301 onto the human skin from the first shower gel tank 105 and the second shower gel tank when the user pushes/activates the first press-button mechanism 109 and the second press-button mechanism respectively.

According to an embodiment herein, the removable brush head 103 is interchangeable for use in different parts of the body like the face, the body, the foot etc. Each removable brush head 103 comprises a plurality of parameters. The plurality of parameters includes the bristle type, size of the removable brush head and the speed at which the removable brush head is rotated.

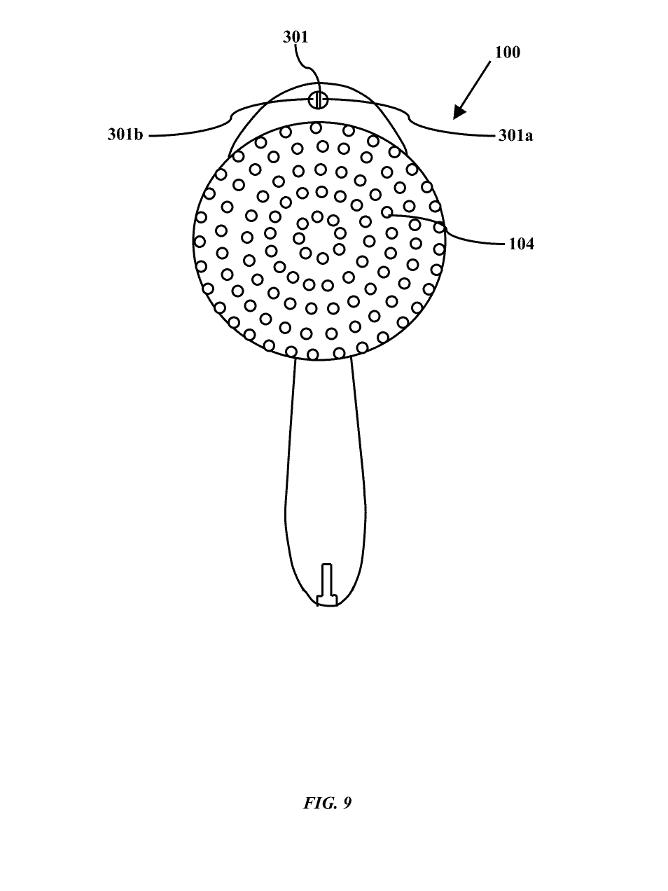

FIG. 9 illustrates a bottom view of the skin polishing apparatus, according to an embodiment herein. A plurality of bristles 104 is projected outwardly from the outer peripheral surface of the removable brush head. The plurality of bristles 104 is placed at a predetermined space from one another. The liquid outlet 301 is attached above the removable brush head at the front portion of the skin polishing apparatus 100. The liquid outlet further comprises a first outlet 301a for dispensing the cleansing liquid and a second outlet 301b for dispensing the perfume. The cleansing liquid and the perfume are dispensed through the first outlet 301a and the second outlet 301b onto the human skin from the first shower gel tank and the second shower gel tank when the user pushes/activates the first press-button mechanism and the second press-button mechanism respectively.

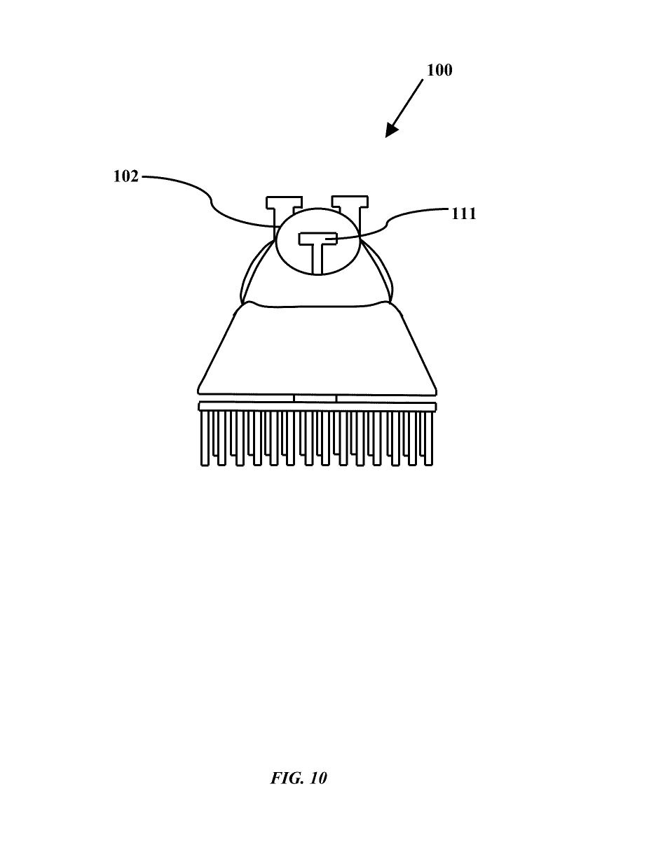

FIG. 10 illustrates a backside view of the skin polishing apparatus, according to an embodiment herein. A charging connector 111 is provided on the back portion of the handle 102 for connecting the skin polishing apparatus 100 to a main supply. The charging connector 111 is externally covered by a silicon seal ring to avoid damage and to block the moisture content from entering into the skin abrading apparatus 100.

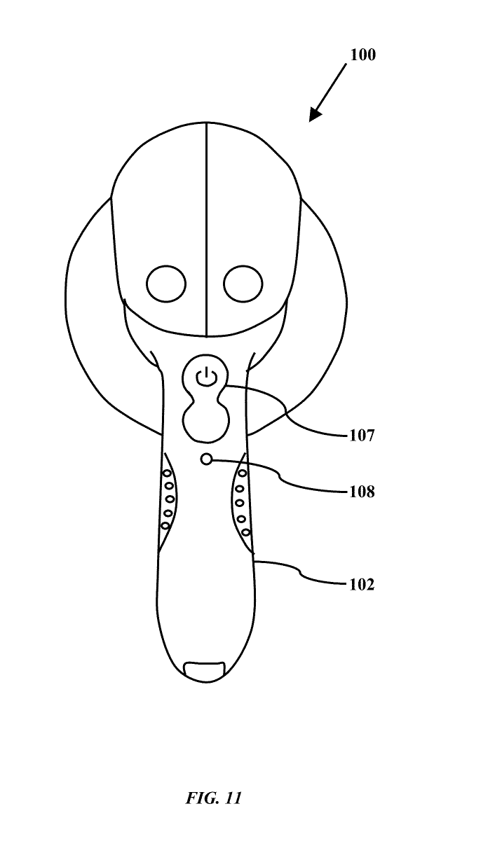

FIG. 11 illustrates a topside view of the skin polishing apparatus, according to an embodiment herein. A power switch 107 is provided on the front portion of the handle 102 for actuating the motor. The power switch 107 has a soft touch outer surface and is internally connected to the PCB. The PCB is electrically coupled to the motor. The user actuates the motor by switching ON the power switch 107 and brings the removable brush head comprising the plurality of bristles into contact with a desired area of the human skin by gripping the handle 107 of the skin polishing apparatus 100 in a hand. After the desired area of the human skin is abraded to clean and remove the dead skin, the dirt and polish the skin, the user deactivates the motor by switching OFF the power switch 107. As soon as the motor is switched OFF, the skin polishing apparatus 100 stops functioning

A power indicator light 108 is provided on the front portion of the handle below the power switch to indicate the status of the skin polishing apparatus 100. The status includes the ON/OFF of the skin polishing apparatus 100 (For example: The power indicator light turns green when the skin polishing apparatus in ON).

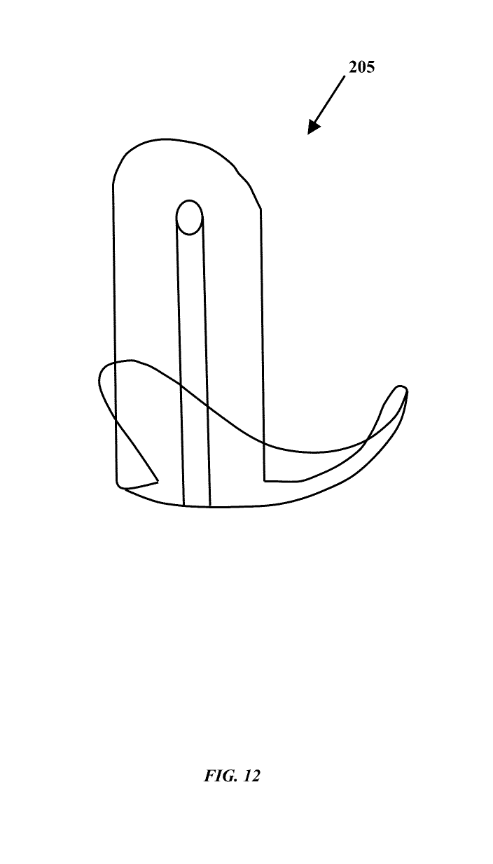

FIG. 12 illustrates a front view of a hanger mounted on a wall for hanging the skin polishing apparatus, according to an embodiment herein. A provision is provided at the centre of the hanger 205 for fixing the hanger 205 to the wall.

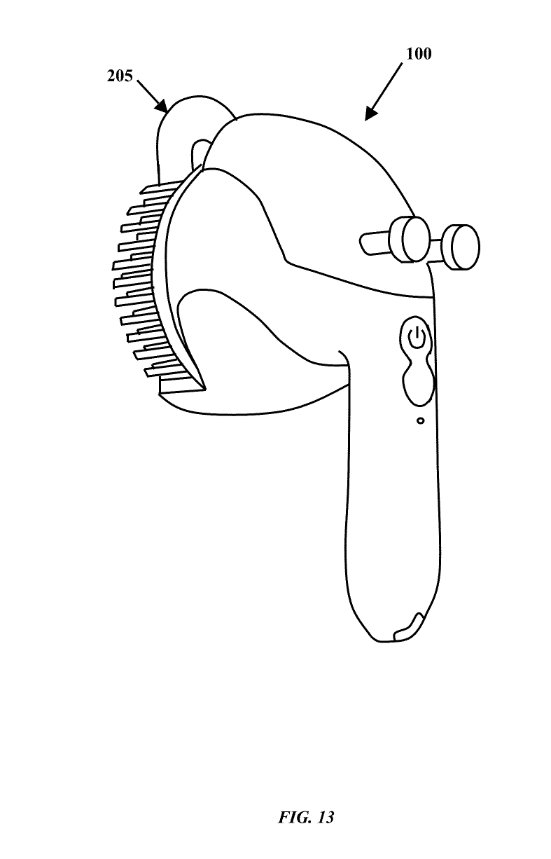

FIG. 13 illustrates a front side perspective view of the skin polishing apparatus hanged to the wall with the help of the hanger, according to an embodiment herein. The skin polishing apparatus 100 is mounted vertically on the hanger 205, when not in use.

According to an embodiment herein, the skin polishing apparatus is placed on the hanger while charging the skin polishing apparatus.

The various embodiments herein provide a method for cleaning and removing the dead skin and the dirt from the human body and polishing the human skin. The method comprises the steps of filling a first shower gel tank with a cleansing liquid, filling a second shower gel tank with a perfume, switching ON the power switch, pressing a first press-button release mechanism for releasing the cleansing liquid onto a human body, pressing a second press-button release mechanism for releasing the perfume onto the human body and running the skin polishing apparatus on the desired areas of the body for removing the dead skin and dirt and also for polishing the skin.

According to an embodiment herein, the user fills the first shower gel tank of the skin polishing apparatus with the cleansing liquid and the second shower gel tank with the perfume respectively. The user brings the skin polishing apparatus into contact with a desired area of the human skin by gripping the handle of the skin polishing apparatus in a hand. The user switches ON the power switch for actuating the skin polishing apparatus. The cleansing liquid and the perfume are dispensed through the first outlet and the second outlet onto the human skin from the first shower gel tank and the second shower gel tank when the user pushes the first press-button mechanism and the second press-button mechanism respectively. The cleansing liquid and the perfume are dispensed from the first outlet and the second outlet while the skin polishing apparatus is rotated to clean and remove the dead skin and dirt and also to polish the skin with an amazing fragrance in the skin. After the desired area of the human skin is abraded, the user deactivates the motor by switching OFF the power switch. As soon as the motor is switched OFF, the skin polishing apparatus stops functioning

The skin polishing apparatus of the embodiments herein provide a method and system for abrading the skin to skin to clean and remove the dead skin and the dirt from the human body. The apparatus of the embodiments herein comprises interchangeable removable brush heads. Each removable brush head has a plurality of parameters. The skin polishing apparatus is water resistive. The skin polishing apparatus comprises two shower gel tanks for filling the desired cleansing gel and perfume respectively and for dispensing the same onto the human skin while abrading the skin.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

What is claimed is:

1. A skin polishing apparatus for cleaning and removing a dead skin and a dirt from a human body and polishing a human skin, the apparatus comprising:
a skin abrading head;
a removable brush head provided at a front portion of the skin abrading head, wherein the removable brush has a plurality of bristles projected outwardly from an outer peripheral surface for abrading a skin;
a first shower gel tank arranged at a back portion of the skin abrading head for storing a cleansing liquid; and
a second shower gel tank installed at the back portion of the skin abrading head for storing a perfume;
a first press-button release mechanism mounted on a top portion of the first shower gel tank for releasing the cleansing liquid;
a second press-button release mechanism mounted on a top portion of the second shower gel tank for releasing the perfume;
a liquid outlet arranged above the removable brush head at the front portion of the skin abrading head for dispensing the cleansing liquid from the first shower gel tank and the perfume from the second shower gel tank respectively; and
a pair of rechargeable batteries for storing and supplying an electrical power to the skin abrading head;
wherein a user fills the cleansing liquid and the perfume in the first shower gel tank and the second shower gel tank respectively and pushes the first press-button and the second press-button to release the cleansing gel and the perfume from the first shower gel tank and the second shower gel tank respectively from a central portion of the removable brush head to clean and remove a dead skin, a dirt from the body and to polish a skin during a rotation of the skin abrading head.

2. The skin polishing apparatus according to claim 1, wherein a handle is integrally formed with a built-in grip for providing a grip to a user.

3. The skin polishing apparatus according to claim 1, wherein a power switch is provided on a front portion of the handle for actuating the skin abrading head.

4. The skin polishing apparatus according to claim 1, wherein a power indicator light is provided on the front portion of the handle below the power switch for indicating a status of the skin abrading head and wherein the status includes an ON/OFF condition of the skin abrading head.

5. The skin polishing apparatus according to claim 1, wherein a charging connector is provided on a back portion of the handle for connecting the skin polishing apparatus to a main supply.

6. The skin polishing apparatus according to claim 1, wherein a printed circuit board (PCB) is mounted along a central axis of the handle and wherein the PCB controls an ON/OFF condition of a motor and a rotatory motion of the removable brush head to a high speed rotation or to a low speed rotation.

7. The skin polishing apparatus according to claim 1, wherein the motor is mounted along a central axis of the handle below the PCB and wherein the motor is driven by a power from the rechargeable batteries.

8. The skin polishing apparatus according to claim 1, wherein a driving gear is mounted along a central axis of the motor for conveying a rotary motion of the motor to a first driven gear and a second driven gear.

9. The skin polishing apparatus according to claim 1, wherein the first driven gear is connected to the driving gear to regulate a rotary speed of a rotator shaft.

10. The skin polishing apparatus according to claim 1, wherein the second driven gear is connected to the driving gear to regulate a rotary speed of a rotator shaft.

11. The skin polishing apparatus according to claim 1, wherein the rotator shaft is extended from the first driven gear to the skin abrading head for actuating the removable brush head.

12. The skin polishing apparatus according to claim 1, wherein the removable brush head is interchangeable and wherein the removable brush head includes a plurality of parameters and wherein the plurality of parameters includes a size, a speed and a bristle type.

13. The skin polishing apparatus according to claim 1, wherein a shape of the removable brush head is any one of a circular shape, an oval shape, an elliptical shape, and a spherical shape.

14. The skin polishing apparatus according to claim 1, wherein the cleansing liquid comprises a skin polishing gel and wherein the skin polishing gel is selected from a group consisting of a shower gel, a deep cleansing gel, a sunscreen lotion, a cream, and a combination thereof

15. The skin polishing apparatus according to claim 1, wherein the first shower gel tank and the second shower gel tank are transparent.

16. The skin polishing apparatus according to claim 1, wherein the skin abrading head has water proof characteristics.

17. The skin polishing apparatus according to claim 1, further comprising an external accessory for placing the apparatus and wherein the external accessory is mounted on a wall and wherein the external accessory is a hanger.

18. A method for cleaning and removing a dead skin and a dirt from a human body and polishing a human skin using a skin polishing apparatus, the method comprising the steps of:
filling a first shower gel tank in a skin abrading head with a cleansing liquid;
filling a second shower gel tank in the skin abrading head with a perfume;
actuating the skin abrading head by switching ON a power switch;
pressing a first press-button mechanism for releasing the cleansing liquid on a skin during a rotation of a rotatable brush head provided at the skin abrading head;
pressing a second press-button mechanism for releasing the perfume on the skin; and
running the skin abrading head in a specific area of the skin for removing a dead skin, dirt and for polishing the skin.
